(19) United States
(12) Patent Application Publication
(10) Pub. No.: US 2018/0174673 A1
(43) Pub. Date: Jun. 21, 2018

(54) Prescription drug management program accountability method and system

(71) Applicants: Edmond Arthur DeFrank, Northridge, CA (US); Allen Mark Jones, Imperial Beach, CA (US); David Clark, Washoe County, NV (US); Nicolas DeFrank, Northridge, CA (US); Antonio DeFrank, Northridge, CA (US)

(72) Inventors: Edmond Arthur DeFrank, Northridge, CA (US); Allen Mark Jones, Imperial Beach, CA (US); David Clark, Washoe County, NV (US); Nicolas DeFrank, Northridge, CA (US); Antonio DeFrank, Northridge, CA (US)

(21) Appl. No.: 15/732,694

(22) Filed: Dec. 16, 2017

Related U.S. Application Data
(60) Provisional application No. 62/435,705, filed on Dec. 16, 2016.

(51) Int. Cl. G16H 20/10; G16H 50/30

(52) U.S. Cl. CPC G16H 20/10; G16H 80/00; G16H 50/30

(57) ABSTRACT

The embodiments disclose a prescription drug management accountability system, including an opioid drug abuse and risk assessment behavioral mental health test integrated within the prescription drug management accountability system for screening a patient that needs an opioid prescription. The prescription drug management accountability system also includes a centralized prescription drug monitoring system that is electronically connected to licensed pharmacies and licensed dispensing entities. The centralized prescription drug monitoring system collects pharmacy prescription information for the patient and produces a report with information on opioid prescriptions of the patient that can be used by a physician with the opioid drug abuse and risk assessment behavioral mental health test before prescribing an opioid.

### CROSS-REFERENCE TO RELATED APPLICATIONS

This application is based on U.S. Provisional Patent Application Ser. No. 62/435,705 filed Dec. 16, 2016; Entitled “PRESCRIPTION DRUG MANAGEMENT PROGRAM METHOD AND SYSTEM”, by DeFrank et al.

### BACKGROUND

Prescription drug abuse is quickly becoming near epidemic due to the lack of complete communications between agencies and companies that deal with the medications themselves and the effects of the abuse. Doctors may be unaware of previous law enforcement incidents involving prescription drugs and the patient they are about to write a prescription for a controlled substance. Illicit drugs are sought through prescriptions then sold to others.

### BRIEF DESCRIPTION OF THE DRAWINGS

FIG. 1A shows for illustrative purposes only an example of a prescription drug management program accountability method and system of one embodiment.

FIG. 1B shows a block diagram of an overview of a prescription drug management program accountability method and system of one embodiment.

FIG. 2 shows a block diagram of an overview of a prescription drug management program accountability method and system data flow of one embodiment.

FIG. 3 shows a block diagram of an overview of patient prescription cash payments triggers a red flag of one embodiment.

FIG. 4 shows a block diagram of an overview flow chart of inputting, analyzing and monitoring testing and prescription activity and results of one embodiment.

FIG. 5 shows a block diagram of an overview flow chart of types of data submissions and displayed information of one embodiment.

FIG. 6A shows for illustrative purposes only an example of PDMPA program physician patient prescription alert of one embodiment.

FIG. 6B shows for illustrative purposes only an example of a PDMPA system screener and opioid behavior assessment of patients with pain of one embodiment.

FIG. 7 shows for illustrative purposes only an example of patient pharmacy records of one embodiment.

FIG. 8A shows for illustrative purposes only an example of patient arrest records of one embodiment.

FIG. 8B shows for illustrative purposes only an example of patient judicial prosecution records of one embodiment.

FIG. 9A shows for illustrative purposes only an example of reversal agent records of one embodiment.

FIG. 9B shows for illustrative purposes only an example of lab results records of one embodiment.

FIG. 9C shows for illustrative purposes only an example of prescriber warning incidents of one embodiment.

FIG. 9D shows for illustrative purposes only an example of dispenser warning incidents of one embodiment.

FIG. 10 shows for illustrative purposes only an example of PDMPA system network of one embodiment.

FIG. 11A shows for illustrative purposes only an example of clinical guidance of one embodiment.

FIG. 11B shows for illustrative purposes only an example of additional clinical guidance of one embodiment.

FIG. 11C shows for illustrative purposes only an example of further clinical guidance of one embodiment.

FIG. 12 shows for illustrative purposes only an example of program administrators PDMPA system network interactions of one embodiment.

FIG. 13 shows for illustrative purposes only an example of law enforcement agency PDMPA system network interactions of one embodiment.

FIG. 14 shows for illustrative purposes only an example of health care industry PDMPA system network interactions of one embodiment.

FIG. 15 shows for illustrative purposes only an example of judicial system PDMPA system network interactions of one embodiment.

FIG. 16 shows for illustrative purposes only an example of interstate cooperative data analysis sharing of one embodiment.

### DETAILED DESCRIPTION OF THE INVENTION

In a following description, reference is made to the accompanying drawings, which form a part hereof, and in which is shown by way of illustration a specific example in which the invention may be practiced. It is to be understood that other embodiments may be utilized and structural changes may be made without departing from the scope of the embodiments.

### General Overview:

It should be noted that the descriptions that follow, for example, in terms of prescription drug management program accountability method and system is described for illustrative purposes and the underlying system can apply to any number and multiple types of physicians and agencies. In one embodiment of the present invention, the prescription drug management program accountability method and system can be configured using manually entered and digitally acquired data. The prescription drug management program accountability method and system can be configured to include multiple agency and entity records and can be configured to include HIPAA patient health records using the embodiments.

In the following descriptions the terms physician, doctor, prescriber and healthcare professional are used interchangeably without any change in meaning. PDMPA are the initials of the Prescription Drug Management Program Accountability method and system and herein are used interchangeably.

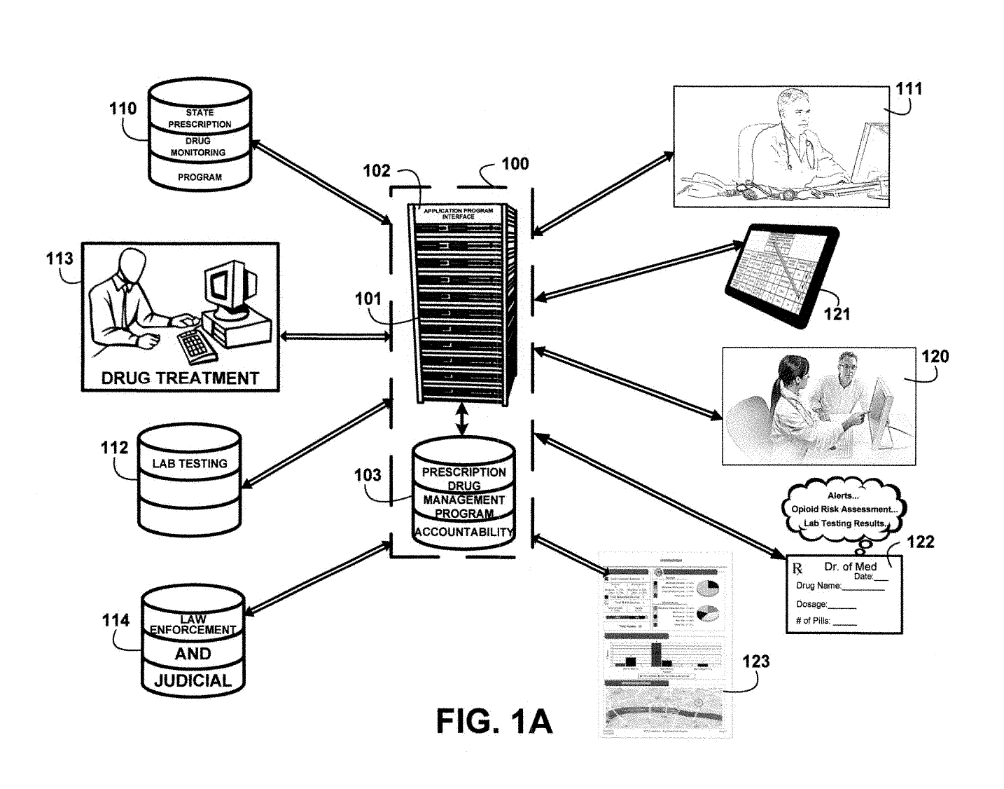

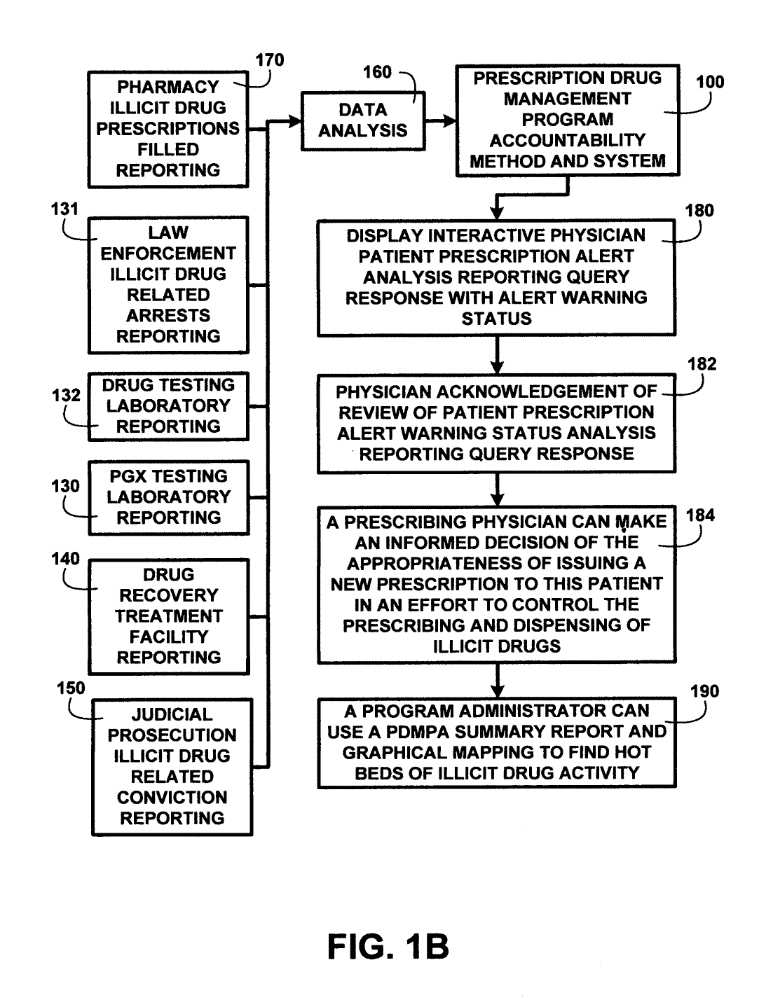

FIG. 1A shows for illustrative purposes only an example of a prescription drug management program accountability method and system of one embodiment. According to the National Institutes of Health (NIH) opioid overdose rates remain high among adults. FIG. 1A shows a prescription drug management program accountability method and system 100 including a network server 101 with an application program interface 102 for compatible connectivity to multiple device operating systems. The network server 101 is coupled to at least one prescription drug management program accountability database 103 creating a centralized external prescription documenting system.

The at least one prescription drug management program accountability database 103 is used to receive data inputted automatically from various entity's databases including a state prescription drug monitoring program database 110. Prescription Drug Monitoring Programs (PDMPs) collect, monitor, and analyze electronically transmitted prescribing and dispensing data submitted by pharmacies and dispensing practitioners. Prescription data is provided only to entities authorized by state law to access the program, such as health care practitioners, pharmacists, regulatory boards and law enforcement agencies. The state prescription drug monitoring program database 110 data is used in a data analysis performed by digital processors in the network server 101.

The at least one prescription drug management program accountability database 103 is used to receive data inputted automatically from lab testing 112 entities databases including from entities that perform blood tests, urine tests, pharmacogenetic PGX tests, drug tests and other testing as physicians deemed appropriate. The lab testing 112 data is used in a data analysis performed by digital processors in the network server 101. The at least one prescription drug management program accountability database 103 is used to receive data inputted automatically from law enforcement and judicial 114 agencies databases. Arrest records and convictions are among the data received and stored on the at least one prescription drug management program accountability database 103. The law enforcement and judicial 114 agencies databases data is used in a data analysis performed by digital processors in the network server 101. Other data can be received by an entity manually entering the data into the prescription drug management program accountability method and system 100. Entities that may manually enter data can include a drug treatment 112 center including half-way houses, rehabilitation clinics, drug recovery center, reversal agencies, and dispensers of drug recovery medications. The data entered by a drug treatment 112 facility is used in a data analysis performed by digital processors in the network server 101. Another group of entities that may manually enter data can include a doctor's office 111. The data entered by a doctor's office 111 is used in a data analysis performed by digital processors in the network server 101.

The prescription drug management program accountability method and system 100 using the data analysis performed by digital processors in the network server 101 provides a physician with data relating to a patient. In one embodiment a physician can review a query for a patient related to past and recent prescriptions, warning incidents that involve arrest and convictions, and lab testing results. The data can for example be displayed on a tablet device in the doctor's office 111. The patient's past and recent prescriptions can be displayed in listing of prescriptions that the data analysis have prepared alerts that indicate warnings that the patient maybe abusing opioids and may be at risk to opioid addiction. While reviewing the data analysis presented the physician can indicate that they have read and have seen the alerts to the warnings and can click a box for a doctor's alert acknowledgement 121. The display can show arrest and convictions related to drugs including opioids, and lab testing results that could indicate current drug use, genetic predispositions to addiction risks. The physician also clicks the corresponding boxes indicating the doctor's alert acknowledgement 121 to those warnings.

The physician can also have the patient take a screener and opioid behavior assessment of patients with pain 120 questionnaires while in the office or examination room. The screener and opioid behavior assessment of patients with pain 120 questionnaires patient responses are transmitted to the network server 101 for data analysis processing. The process transmits the results of the data analysis to the physician for further information to allow the physician to evaluate any warnings of the patient's drug risk assessment.

The prescription drug management program accountability method and system 100 provides a means for a prescribing physician considering alerts, opioid risk assessment and lab testing results to make an informed decision of the appropriateness of issuing a new prescription to a patient in an effort to control the prescribing and dispensing of illicit drugs 122.

The prescription drug management program accountability method and system 100 can also provide data analysis and warnings to pharmacies and drug dispensers to make an informed decision of the appropriateness of filling a prescription that may provide the patient with an excess of an opioid drug that can be sold.

The prescription drug management program accountability method and system 100 can also provide data analysis and warnings to law enforcement agencies in for example a summary report and graphical mapping to find hot beds of illicit drug activity 123 to facilitate the investigation and apprehension of those involved in illicit drug activities of one embodiment.

The prescription drug management program accountability method and system 100 provides a physician with fast response comprehensive information and clinical guidance allowing the physician to be informed prior to prescribing an opioid medication to a patient who may be at a high risk of becoming addicted or a patient that is clearly already addicted and needs treatment for that addiction. The prescription drug management program accountability method and system 100 provides dispensers of opioid drugs information and data analysis to better identify potential opioid abuse and prevent the distribution of additional opioid medications to the illicit trade. It also provides law enforcement with credible facts data analysis that may lead to the sources of a local opioid addiction and trafficking operation that is fueling the opioid drug problems currently engulfing many of our communities.

FIG. 1B shows a block diagram of an overview of a prescription drug management program accountability method and system of one embodiment. FIG. 1 shows a prescription drug management program accountability method and system 100 that includes communications of a patient's illicit drug prescription activities from prescribers, dispensers and agencies that deal with illegal abuse of those prescription drugs. The prescription drug management program accountability method and system 100 receives patient information from pharmacy illicit drug prescriptions filled reporting 170, law enforcement illicit drug related arrests reporting 131, drug testing laboratory reporting 132, PGX testing laboratory reporting 130, drug recovery treatment facility reporting 140, and judicial prosecution illicit drug related conviction reporting 150.

The prescription drug management program accountability method and system 100 automatically performs an integration of the reported patient information to insure the data is correctly identified to the same person. The prescription drug management program accountability method and system 100 automatically performs a data analysis 160 using proprietary algorithms to identify warning red flags of prescription drug abuse. The abuse may be overlapping prescriptions from different physicians providing the patient with too many doses within the prescribed period of days. Red flags may take the form of prescription drug payment using insurance and then cash in an attempt to conceal the purchase of multiple prescriptions. The warning incidents are provided to a registered patient data requestor making a data query.

The registered patient data requestor can be a program administrator, a physician, and a law enforcement agency. The patient data is shown on a display interactive physician patient prescription alert analysis reporting query response with alert warning status 180. Before making a new prescription the physician acknowledgement of review of patient prescription alert warning status analysis reporting query response 182 is performed using the interactive features of the display. The prescription drug management program accountability method and system 100 provides a prescribing physician with a complete analysis of the patient's prescription drug use and abuse in order to make an informed decision of the appropriateness of issuing a new prescription to this patient. A prescribing physician can make an informed decision of the appropriateness of issuing a new prescription to this patient in an effort to control the prescribing and dispensing of illicit drugs 184. A program administrator can use a PDMPA summary report and graphical mapping to find hot beds of illicit drug activity 190 of one embodiment.

The registered patient data requestor for example a State Government program administrator can use the prescription drug management program accountability method and system 100 to request various high to low level reports. For example a summary report and graphical mapping of the number of red flag warnings across the state to find hot beds of illicit drug activity. Using the arrest and conviction reporting an overlay graphical map can identify areas where the numbers of illicit drug arrest and prescription red flags overlap. Once those overlapping areas have been identified the program administer can request a finer more detailed report by for example zip codes in those hot bed areas.

A report that correlates arrests and convictions with prescribers for example may expose those doctors that have a much greater percentage than the average of patients that have a large number of red flags and how many of those patients have arrest and convictions related to illicit drugs. Law enforcement can use the reports available from the prescription drug management program accountability method and system 100 possible suspects in illicit drug sales by ranking patients based on a number of findings for example abnormally high red flag warnings for overlapping prescriptions that produce quantities of for example opioid pain killers that greatly exceed the prescribed dosage.

Both State program administrators and law enforcement can investigate joint efforts with a neighboring state where the hot bed areas are along the mutual state Borders. For example high arrest red flag warnings in one state a high pharmacy prescription dispensing of illicit drugs across the border in the adjoining state.

A physician can request reports from the prescription drug management program accountability method and system 100 to search a patient's EMR for previous addiction problems of a patient who is demanding a greater prescription frequency or refills. For example the physician can see on the PDMPA interactive display that this patient has a high number of red flag warnings. Should the patient's EMR (electronic medical records) show previous addiction incidents the physician may decide to reevaluate testing and alternative non-illicit drugs prescriptions and therapy treatment to lessen the continuation of an addictive posture or recurrence of a previous addiction. The PDMPA can analyze the outcome of screener and opioid assessment for patients with pain of possible opioid abuse in chronic pain patients from EMR records and any correlation to red flag warnings. The physician can compare the subject patient's red flag warnings and see how the data may justify the subject patient having a SOAPP-R evaluation. The physician can request a report on other prescribers that are writing prescriptions for the same or similar illicit drugs to the subject patient. This may indicate an addiction.

The PDMPA analysis of prescription illicit drug prescribing and pharmacy dispensing of those prescriptions can be utilized to assist physicians in identifying potential patient abusers and program administrators and law enforcement to identify potential offenders including patients, doctors and pharmacies and abuse high-level locations that would not be possible without the cross analysis of the reported data from seemingly non-related groups and agencies. The expanded opportunities afforded by the PDMPA can help reduce the cost in dollars, human suffering and violent crimes that accompany illicit drug abuse activities.

### DETAILED DESCRIPTION

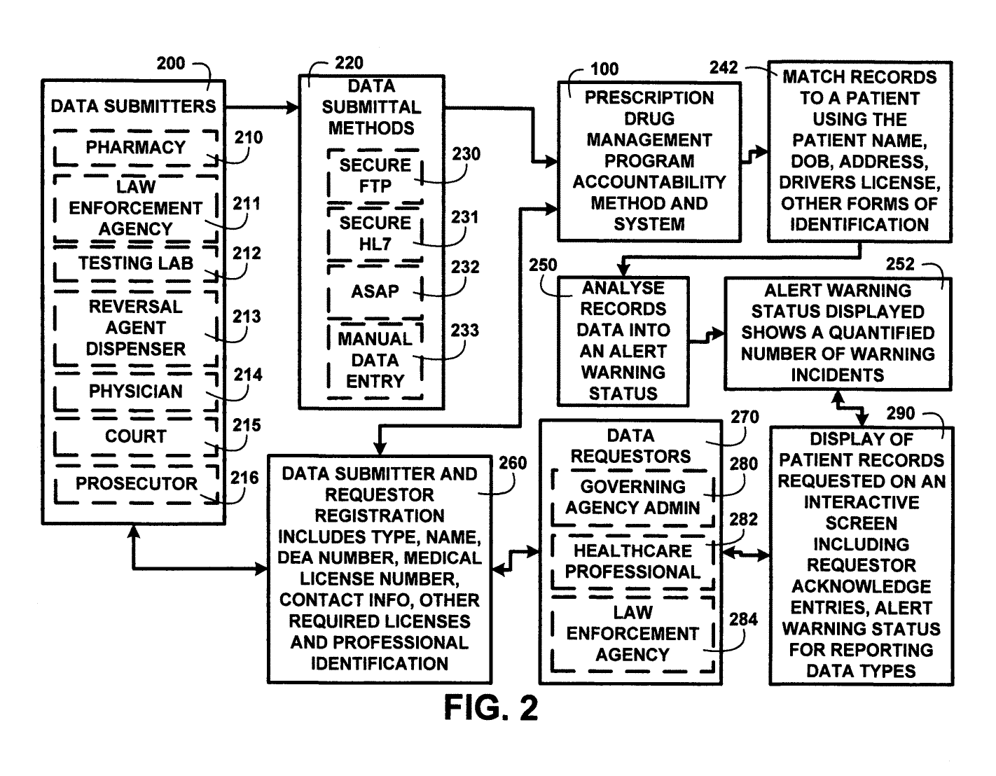

FIG. 2 shows a block diagram of an overview of a prescription drug management program accountability method and system data flow of one embodiment. FIG. 2 shows data submitters 200 who are reporting and includes a pharmacy 210, law enforcement agency 211, testing lab 212, reversal agent dispenser 213, physician 214, court 215 and prosecutor 216. The data submitters 200 can use data submittal methods 220 including a secure FTP 230, secure HL7 231, ASAP 232 and manual data entry 233.

The prescription drug management program accountability method and system 100 includes multiple processes to match records to a patient using the patient name, DOB, address, driver's license, and other forms of identification 242. The prescription drug management program accountability method and system 100 includes multiple processes to analyze records data into an alert warning status 250. The alert warning status displayed shows a quantified number of warning incidents 252. The alert warning status is a part of the display of patient records requested on an interactive screen and includes requestor acknowledge entries, alert warning status for reporting data types 290. Data requestors 270 can include a governing agency admin 280, a healthcare professional 282 and a law enforcement agency 211.

To gain access to the reported patient data, data submitters and data requestors must register and be approved for access. Data submitter and requestor registration includes type, name, DEA number, medical license number, contact info, other required licenses and professional identification 260 of one embodiment.

### Illicit Drug Alert Status Warning and Prescriber Accountability System:

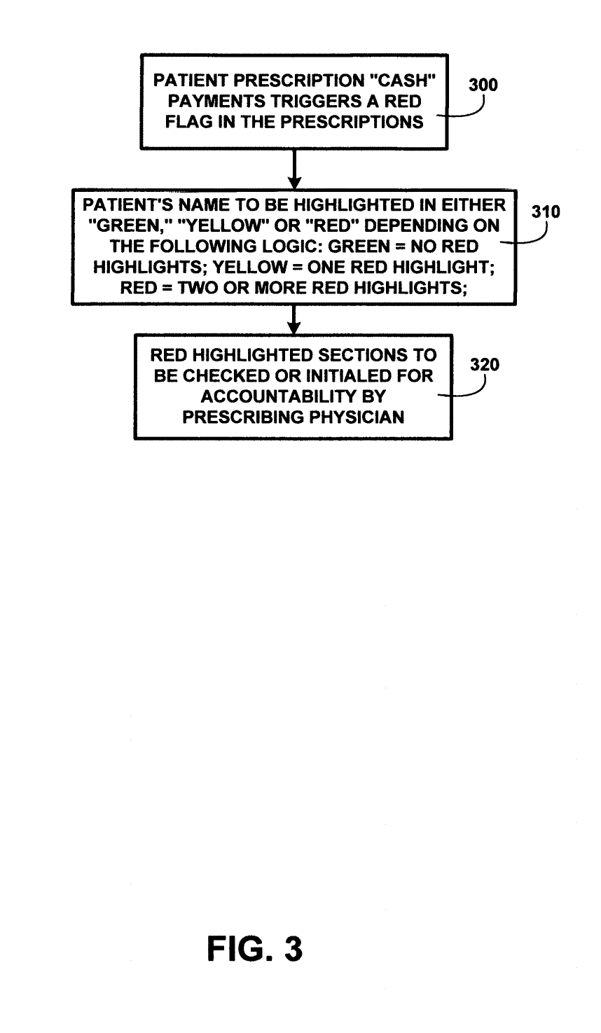

FIG. 3 shows a block diagram of an overview of patient prescription cash payments triggers a red flag of one embodiment. FIG. 3 shows patient prescription “cash” payments triggers a red flag in the prescriptions 300 activities. Red flag analysis is used to create the alert warning status. In addition to text and data of activities a patient's name to be highlighted in either “green,” “yellow” or “red” depending on the following logic: green=no red highlights; yellow=one red highlight; red=two or more red highlights 310. Red highlighted sections to be checked or initialed for accountability by prescribing physician 320 of one embodiment.

### Analyzing and Integrating Data Submittals:

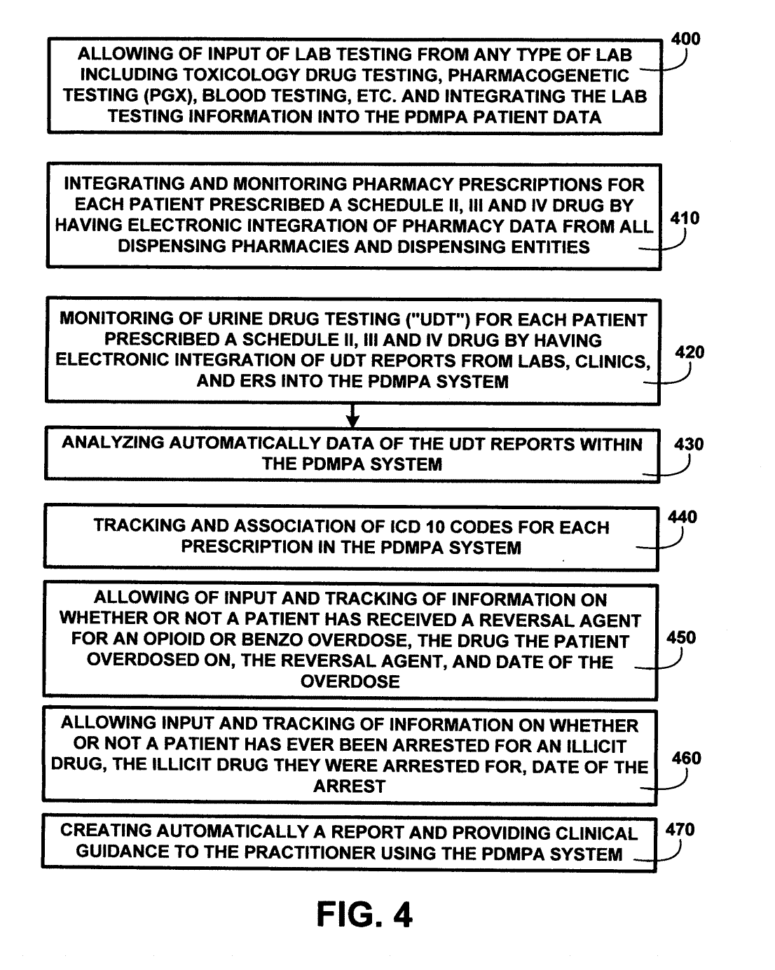

FIG. 4 shows a block diagram of an overview flow chart of inputting, analyzing and monitoring testing and prescription activity and results of one embodiment. FIG. 4 shows allowing of input of lab testing from any type of lab including toxicology drug testing, pharmacogenetic testing (PGX), blood testing, etc. and integrating the lab testing information into the PDMPA patient data 400 of one embodiment.

The PDMPA processing includes integrating and monitoring pharmacy prescriptions for each patient prescribed a schedule II, III and IV drug by having electronic integration of pharmacy data from all dispensing pharmacies and dispensing entities 410. The PDMPA processing includes monitoring of urine drug testing (“UDT”) for each patient prescribed a schedule ii, iii and iv drug by having electronic integration of UDT reports from labs, clinics, and ERS into the PDMPA system 420. The PDMPA processing includes analyzing automatically data of the UDT reports within the PDMPA system 430. The PDMPA processing includes tracking and association of ICD 10 codes for each prescription in the PDMPA system 440.

The PDMPA processing includes allowing of input and tracking of information on whether or not a patient has received a reversal agent for an opioid or Benzo overdose, the drug the patient overdosed on, the reversal agent, and date of the overdose 450. The PDMPA processing includes allowing input and tracking of information on whether or not a patient has ever been arrested for an illicit drug, the illicit drug they were arrested for, date of the arrest 460. The PDMPA processing includes creating automatically a report and providing clinical guidance to the practitioner using the PDMPA system 470. The clinical guidance can include education on how to interpret patient data based on the patient past and current behavior, for example past arrest for an illicit drug, and filling of opioid prescriptions from multiple prescribers, paying cash and other indications of a drug abuse behavior of one embodiment.

### Types of Data Submissions:

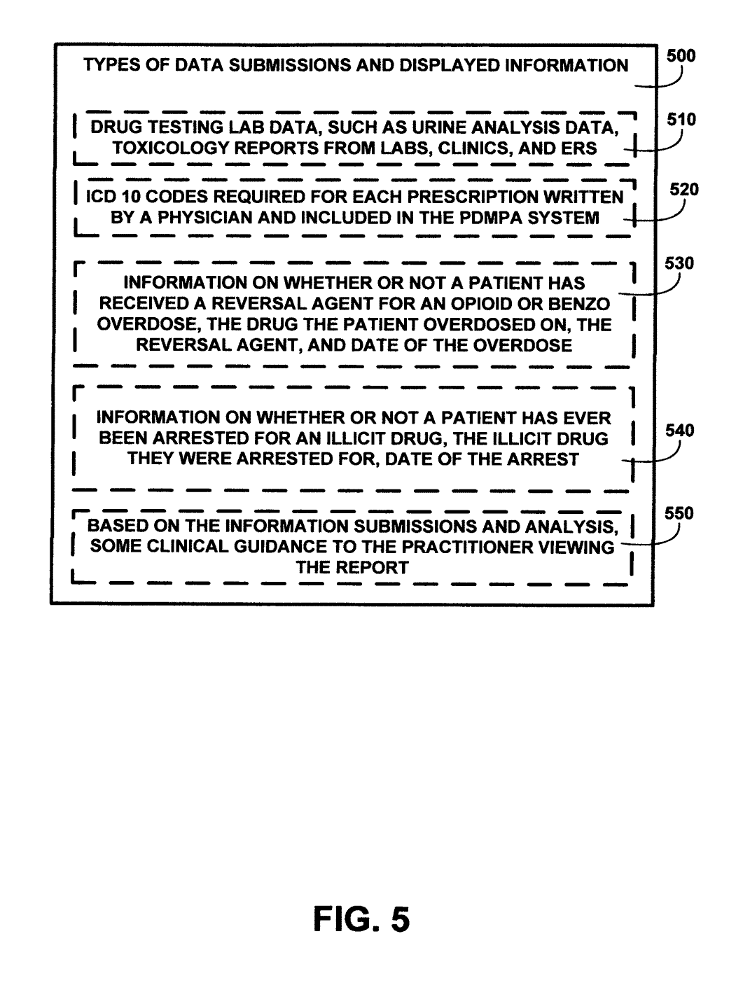

FIG. 5 shows a block diagram of an overview flow chart of types of data submissions and displayed information of one embodiment. FIG. 5 shows types of data submissions and displayed information 500. Drug testing lab data, can include urine analysis data, toxicology reports from labs, clinics, and ERS 510. ICD 10 codes required for each prescription written by a physician and included in the PDMPA system 520. Information on whether or not a patient has received a reversal agent for an opioid or Benzo overdose, the drug the patient overdosed on, the reversal agent, and date of the overdose 530. Information on whether or not a patient has ever been arrested for an illicit drug, the illicit drug they were arrested for, date of the arrest 540. Based on the information submissions and analysis, some clinical guidance to the practitioner viewing the report 550 can be included in a PDMPA interactive display of one embodiment.

Clinical guidance to the practitioner viewing the report can be included in a PDMPA interactive display can include some education on the opioid addict susceptible classifications, behaviors and drug use patterns. The clinical guidance can include patterns of drug use including opioids leading to a heroin addiction. The clinical guidance can include education on the significance of genetic and toxicology reported conditions that serve as red flags to probable opioid addiction to better equip the practitioner in evaluating the patient they are treating or a patient being scheduled for a procedure for which the practitioner would prescribe an opioid pain killer.

### Physician Patient Data Request Response Interactive Display:

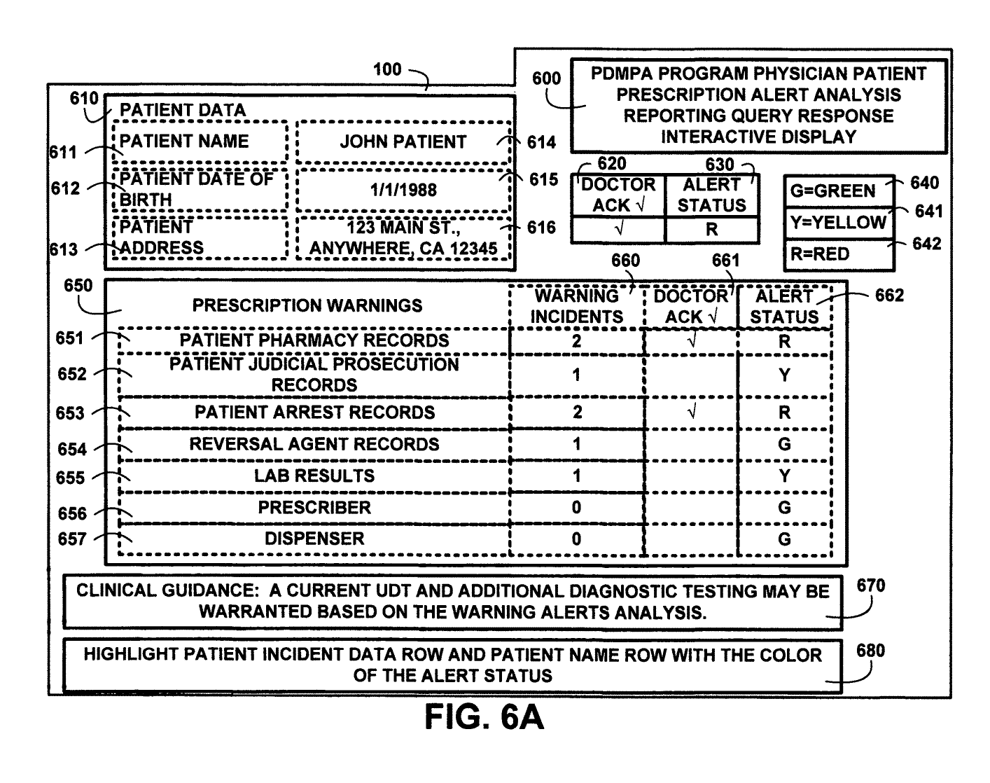

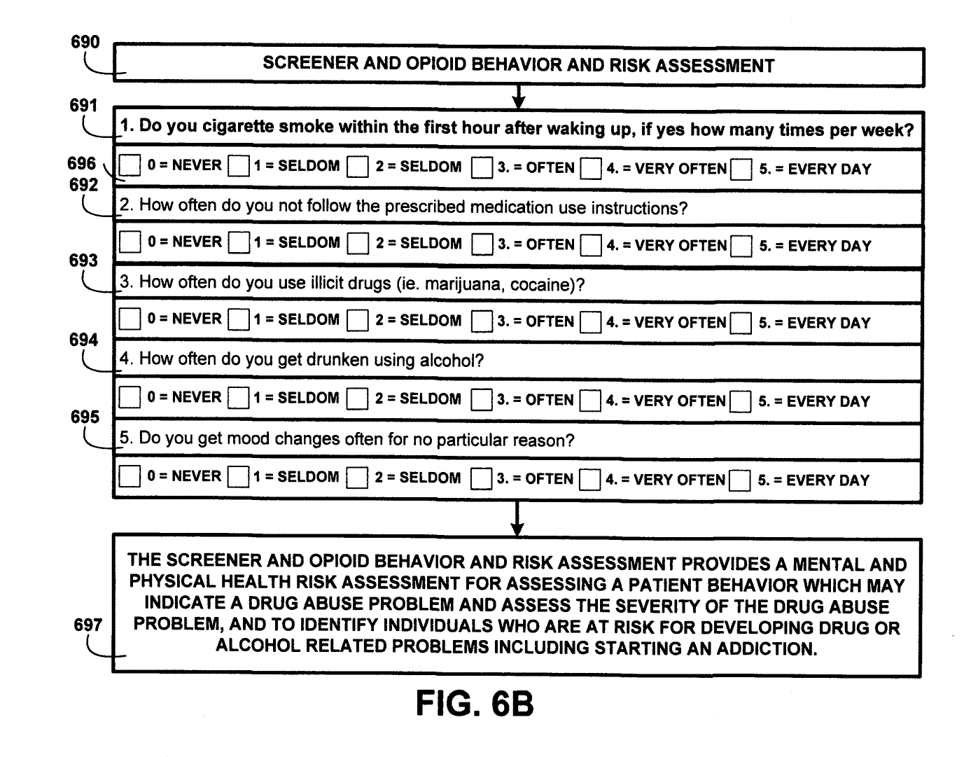

FIG. 6A shows for illustrative purposes only an example of PDMPA program physician patient prescription alert of one embodiment. FIG. 6A shows a PDMPA program physician patient prescription alert analysis reporting query response interactive display 300. In this example of a query response interactive display patient data including a patient name 611 for example john patient 614, a patient date of birth 612 for example 1/1/1988 615, and a patient address 613 for example 123 Main St., Anywhere, CA 12345 616.

The query response interactive display includes prescription warnings 650 for reported data types and includes a number of warning incidents 660, a Doctor ack ℏ 661 and an alert status 662. A Doctor ack ℏ 661 is a Doctor acknowledgement that they have reviewed the alert status 662 data and is a process in creating accountability for prescription of an illicit drug to a particular patient. The Doctor using the interactive features Doctor ack ℏ 620 can input a ℏ mark manually and using a drop down selection. The alert status 630 feature can include a letter to qualify the status for example an R. The alert status qualifier letters include G=Green 640, Y=Yellow 641 and R=Red 642 of one embodiment.

In this example a row can include patient pharmacy records 651, 2, ℏ, R; patient judicial prosecution records 652, 1, no check mark, Y; patient arrest records 653, 2, ℏ, R; reversal agent records 654, 1, no check mark, G; lab results 655, 1, no check mark, Y; prescriber 656, 0, no check mark, G; dispenser 657, 0, no check mark, G. The prescribing physician can review for example an automated clinical guidance: a current UDT and additional diagnostic testing may be warranted based on the warning alerts analysis 670. To assist the physician in discerning an alert status the PDMPA interactive display will highlight patient incident data row and patient name row with the color of the alert status 680 indicated of one embodiment.

### PDMPA System Screener and Opioid Behavior and Risk Assessment:

FIG. 6B shows for illustrative purposes only an example of a PDMPA system screener and opioid behavior assessment of patients with pain of one embodiment. FIG. 6B shows a screener and opioid behavior and risk assessment 690 patient questionnaire. The screener and opioid behavior and risk assessment 690 patient questionnaire is used for assessing a patient behavior which may indicate a drug abuse problem and assess the severity of the drug abuse problem, and to identify individuals who are at risk for developing drug or alcohol-related problems including starting an addiction 697. A physician can use the PDMPA system to download the screener and opioid behavior and risk assessment 690 patient questionnaire during an examination. The physician can ask the patient the questions and mark the patient's response or using a touch screen digital device for example a tablet have the patient mark the responses themselves.

In one embodiment the screener and opioid behavior and risk assessment 690 patient questionnaire can include the following assessment questions including 1. Do you cigarette smoke within the first hour after waking up, if yes how many times per week? 691. A patient's responses can include 0=never, 1=seldom, 2=seldom, 3. =often, 4. =very often, and 5. =every day 696 which they can indicate by clicking on the corresponding box in front of the response. Additional question can include 2. How often do you not follow the prescribed medication use instructions? 692, 3. How often do you use illicit drugs (i.e. marijuana, cocaine)? 693, 4. How often do you get drunken using alcohol? 694, and 5. Do you get mood changes often for no particular reason? 695.

The patient responses are transmitted to the PDMPA system network where the patients file is updated to include the screener and opioid behavior and risk assessment 690 patient questionnaire responses. The screener and opioid behavior and risk assessment 690 patient questionnaire can be adapted to ask additional or differing questions periodically for example for the same patient at subsequent visits to the physician including visits to a different physician of one embodiment.

### Patient Pharmacy Records:

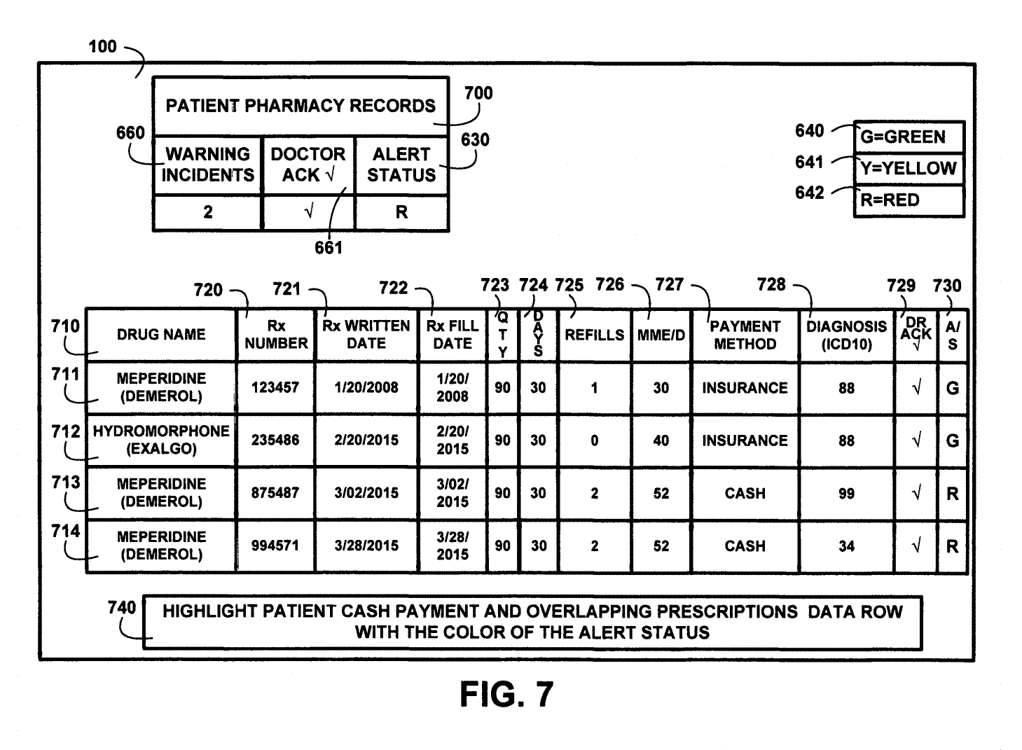

FIG. 7 shows for illustrative purposes only an example of patient pharmacy records of one embodiment. FIG. 7 shows patient pharmacy records 653 including a summary of warning incidents 660 2 700, the doctor ack ℏ 661 may include a ℏ 702 mark by the doctor, and an alert status 663 letter R following the alert status symbols of G=Green 640, Y=Yellow 641, and R=Red 642. The patient pharmacy records 653 include for example a drug name 710, Rx number 720, Rx written date 721, Rx fill date 722, Qty 723 meaning Quantity, Days 724, Refills 725, MME/D 726 meaning Morphine Milligram Equivalents per Day, payment method 727, diagnosis (ICD10) 728, Dr. ack ℏ 729 and patient pharmacy records A/S 730 meaning Alert Status.

In this example the rows list prescriptions including the drug name 710 in patient pharmacy records row #1 711 meperidine (Demerol), 123457, 1/20/2008, 1/20/2008, 90, 30 1, 30, insurance, 88, ℏ, G; patient pharmacy records row #2 712 hydromorphone (Exalgo), 235486, 2/20/2015, 2/20/2015, 90, 30, 0, 40, insurance, 88, ℏ, G; patient pharmacy records row #3 713 meperidine (Demerol), 875487, 3/02/2015, 3/02/2015, 90, 30, 2, 52, cash, 99, ℏ, R; and patient pharmacy records row #4 meperidine (Demerol), 994571, 3/28/2015, 3/28/2015, 90, 30, 2, 52, cash, 34, ℏ, R. To assist the physician in discerning an alert status the PDMPA interactive display will highlight patient cash payment and overlapping prescriptions data row with the color of the alert status 740 of one embodiment.

### Patient Arrest Records:

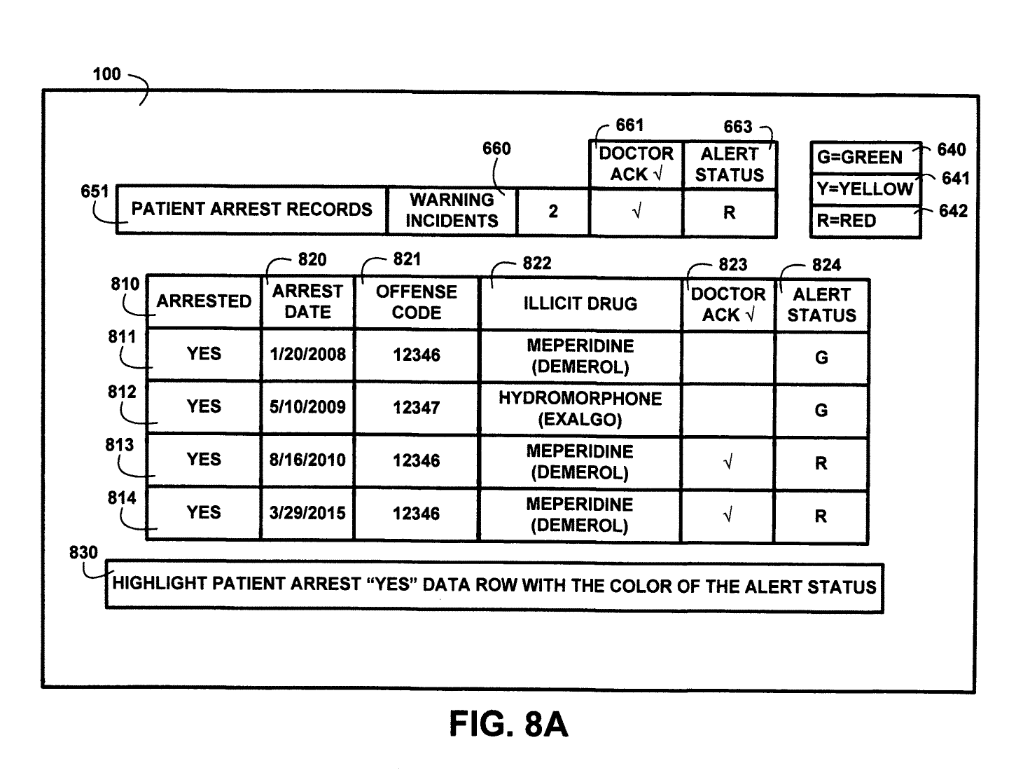

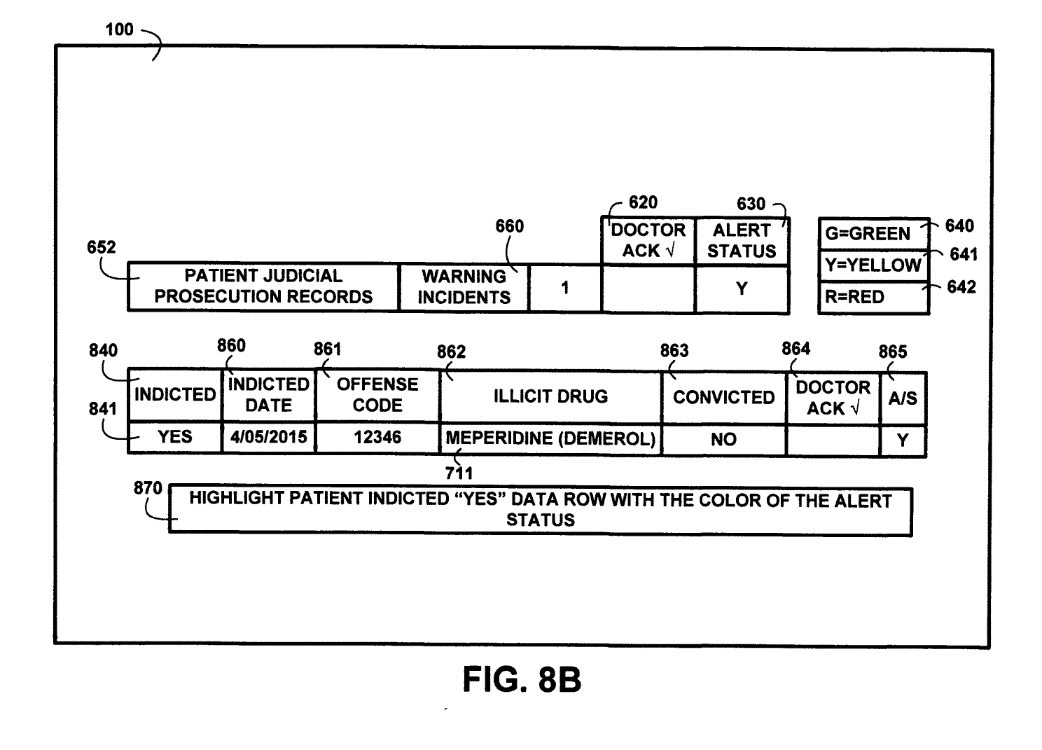

FIG. 8A shows for illustrative purposes only an example of patient arrest records of one embodiment. FIG. 8A shows patient arrest records 651 and includes a number of warning incidents 660 for example 2, a Doctor ack ℏ 661 shown as including a ℏ mark, and an alert status 663 shown as including an R. The alert status column will include a letter following the alert status symbols G=Green 640, Y=Yellow 641 and R=Red 642 of one embodiment.

The patient arrest records 651 will include entries for arrested 810, arrest date 820, offense code 821, illicit drug 822, patient arrest records Doctor ack ℏ 823, and patient arrest records alert status 824. In this example patient arrest records row #1 811 can include yes, 1/20/2008. 12346, meperidine (Demerol), ℏ, G; patient arrest records row #2 812 yes, 5/10/2009, 12347, hydromorphone (Exalgo), ℏ, G; patient arrest records row #3 813 yes, 8/16/2010, 12346, meperidine (Demerol), ℏ, R; and patient arrest records row #4 814 yes, 3/29/2015, 12346, meperidine (Demerol), ℏ, R. To assist the physician in discerning an alert status the PDMPA interactive display will highlight patient arrest “yes” data row with the color of the alert status 830 of one embodiment.

### Patient Judicial Prosecution Records:

FIG. 8B shows for illustrative purposes only an example of patient judicial prosecution records of one embodiment. FIG. 8B shows patient judicial prosecution records 652 including a summary of warning incidents 660 for example 1, doctor ack ℏ 620 shown as blank and an alert status 630 shown including a Y. The alert status column will include a letter following the alert status symbols G=Green 640, Y=Yellow 641 and R=Red 642. The patient judicial prosecution records 652 for example can include Indicted 840, indicted date 860, offense code 861, illicit drug 862, convicted 863, patient judicial prosecution records Doctor ack ℏ 864, and patient judicial prosecution records alert status 865. In this example patient judicial prosecution records row #1 841 can include yes, 4/05/2015, 12346, meperidine (Demerol), no, blank, Y of one embodiment.

### Reversal Agent Records:

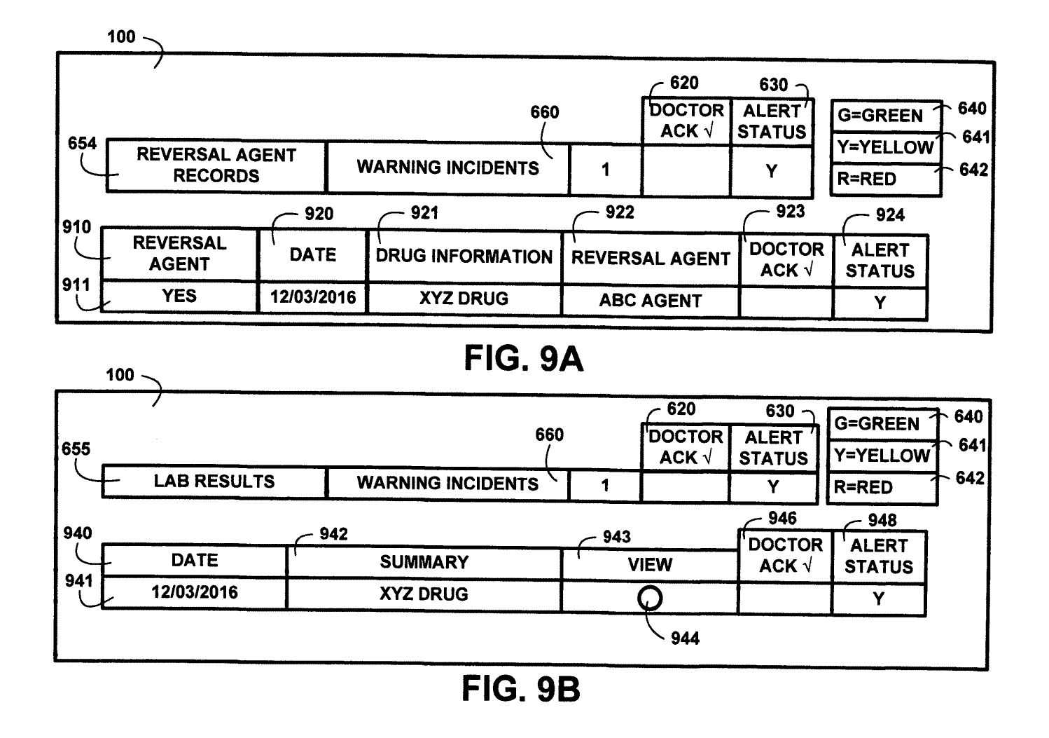

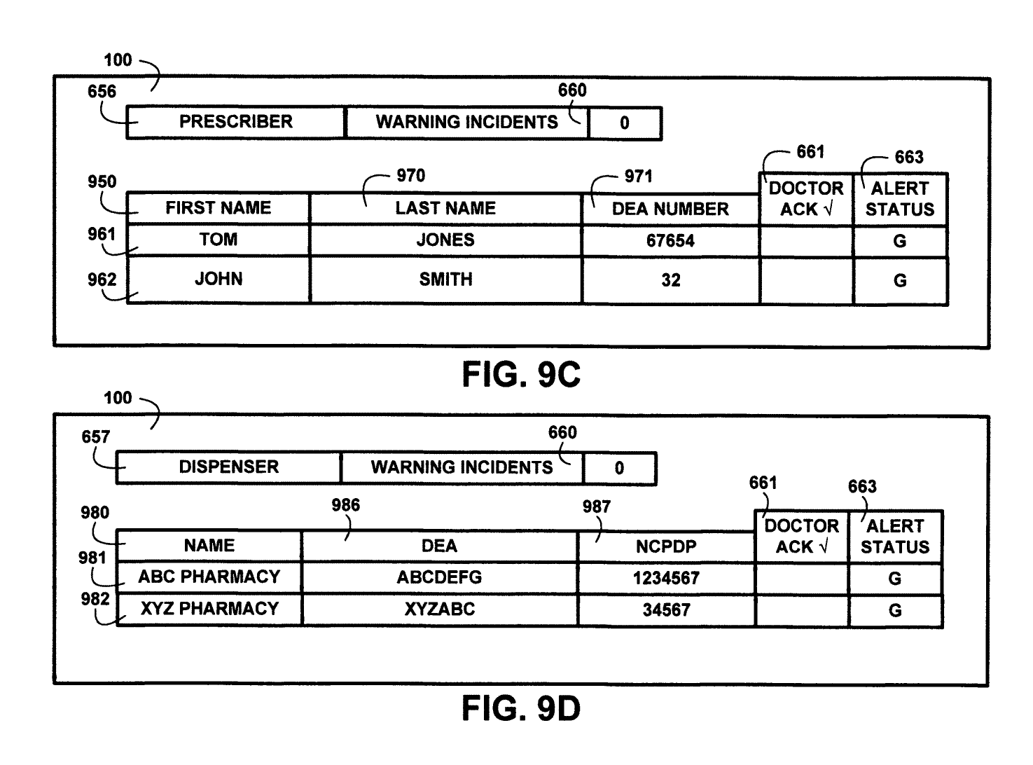

FIG. 9A shows for illustrative purposes only an example of reversal agent records of one embodiment. FIG. 9A shows reversal agent records 654 management program physician patient prescription alert analysis reporting query response interactive display of reversal agent records reporting. The reversal agent records 654 show analysis results of warning incidents 660 for example 1, doctor ack ℏ 620 shown as blank and an alert status 630 shown including a Y. The alert status column will include a letter following the alert status symbols G=Green 640, Y=Yellow 641 and R=Red 642 of one embodiment.

The display lists patient record data including is reversal agent 910, date 920, drug information 921, reversal agent 922, doctor ack ℏ 923, and alert status 924. Queries of all records provide an interactive physician doctor ack ℏ where the requesting physician enters a check mark using a stylist, a drop down selection, and other methods of acknowledging they have reviewed the report data and alert status analysis results. For example the record data shows reversal agent records row #1 911 yes, 12/03/2016, the drug information xyz drug, and reversal agent for example abc agent. The physician leaves the acknowledge box blank since in this example the alert status indicates Y for yellow of one embodiment.

### Lab Results Records:

FIG. 9B shows for illustrative purposes only an example of lab results records of one embodiment. FIG. 9B shows lab results data and includes the number of warning incidents for example 1. The lab results data is listed by date, a summary, and a view button to activate a link to view the printed lab results details. The lab result listing includes a doctor ack ℏ and an alert status. In this example the single row can include 12/03/2016, xyz drug, viewing button, blank, Y. The viewing button when pushed or tapped queries the lab results which are then displayed on the interactive screen of one embodiment.

### Prescriber Warning Incidents:

FIG. 9C shows for illustrative purposes only an example of prescriber warning incidents of one embodiment. FIG. 9C shows prescriber data and warning incidents for example 0. The prescriber data includes first name, last name, DEA number, doctor ack ℏ and alert status. In this example the first row includes Tom, Jones, 67654, blank, G. The second row includes John, Smith, 32, blank, G of one embodiment.

### Dispenser Warning Incidents:

FIG. 9D shows for illustrative purposes only an example of dispenser warning incidents of one embodiment. FIG. 9D shows dispenser data and warning incidents for example 0. The dispenser data includes Name, DEA, NCPDP, doctor ack and alert status. The first row includes for example abc pharmacy, abcdefg, 1234567, blank, G. The second row includes for example xyz pharmacy, xyzabc, 34567, blank, G of one embodiment.

### PDMPA System Network:

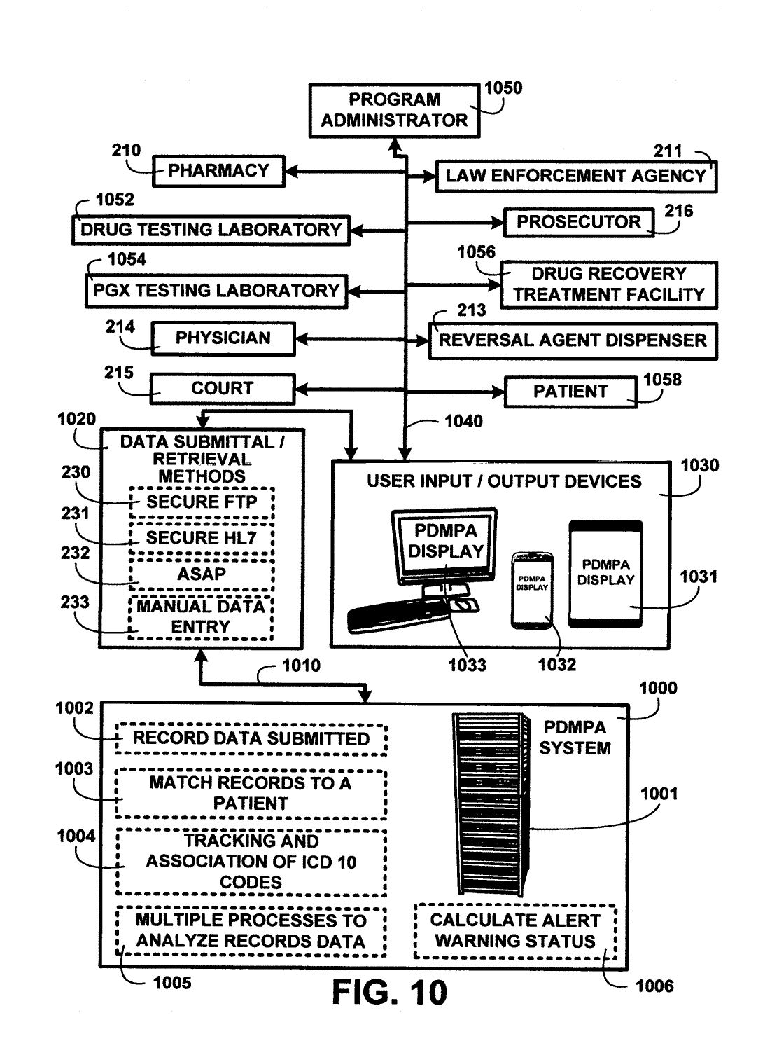

FIG. 10 shows for illustrative purposes only an example of PDMPA system network of one embodiment. FIG. 10 shows the program administrator 1050 of the PDMPA system 1000. The program administrator 1050 uses the PDMPA system 1000 and PDMPA system network 1001 to gather data from various entities relating to drug abuse and criminal activities. The various entities include at least one of each of the following including pharmacy 210, drug testing laboratory 1052, PGX testing laboratory 1054, physician 214, court 215, law enforcement agency 211, prosecutor 216, drug recovery treatment facility 1056, reversal agent dispenser 213, and patient 1058.

Digital data communications connections 1040 are made using user input/output devices 1030 including a computer 1033, smart phone 1032 and tablet 1031. Data submittal/retrieval methods 1020 include secure FTP 230, secure HL7 231, ASAP 232, and manual data entry 233. The PDMPA system 1000 uses the PDMPA system network 1001 to record data submitted 1002, match records to a patient 1003, tracking and association of ICD 10 codes 1004, multiple processes to analyze records data 1005, and calculate alert warning status 1006 of one embodiment.

### Clinical Guidance:

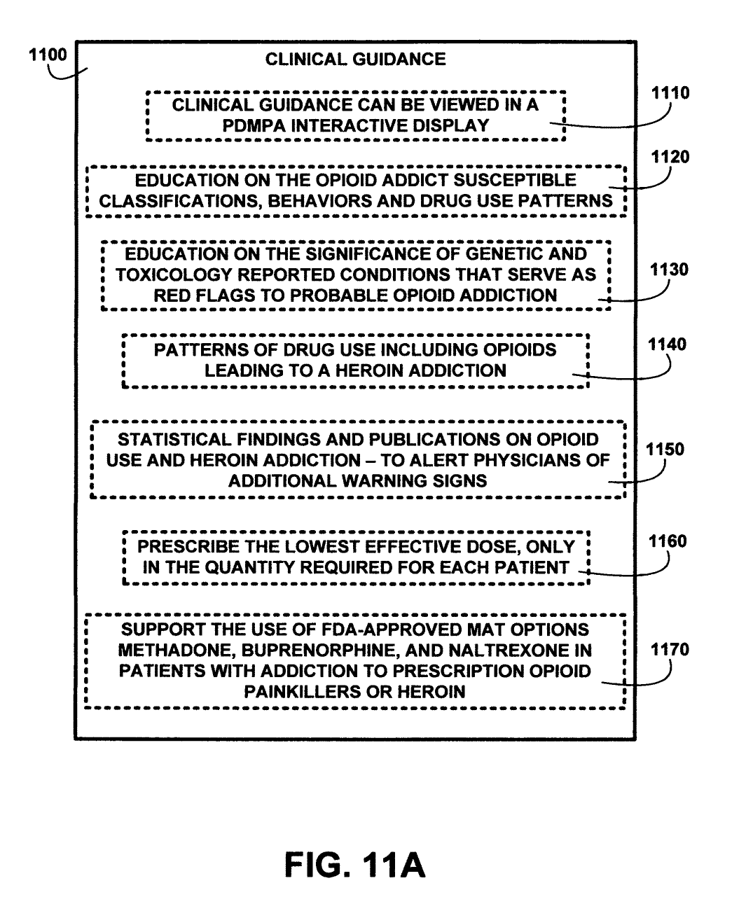

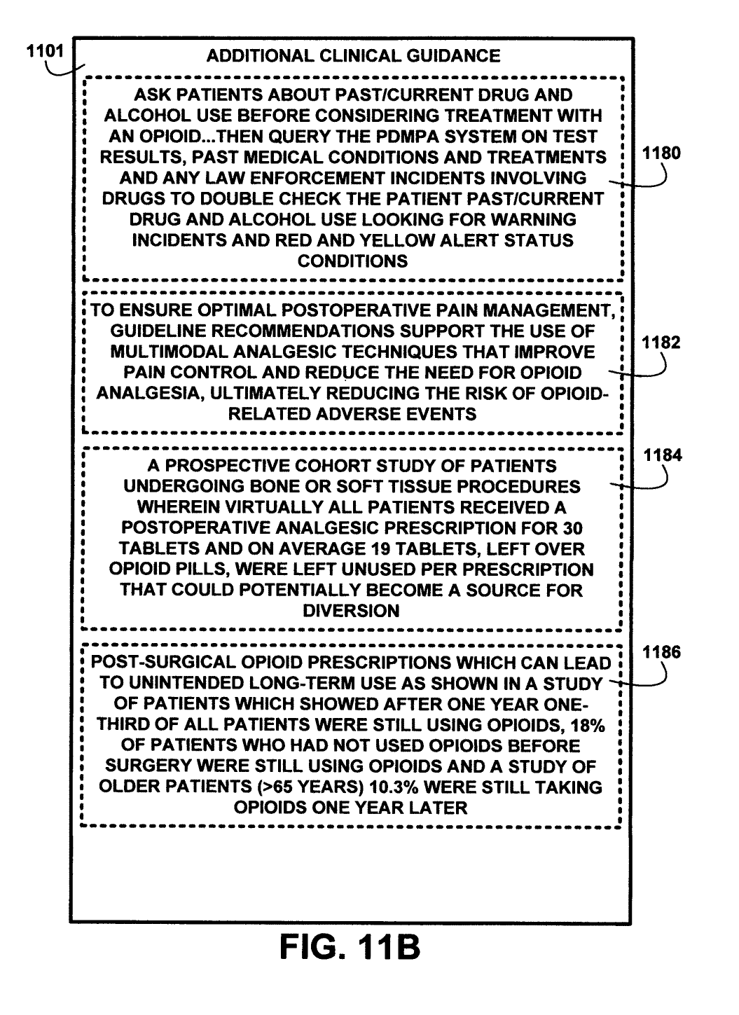

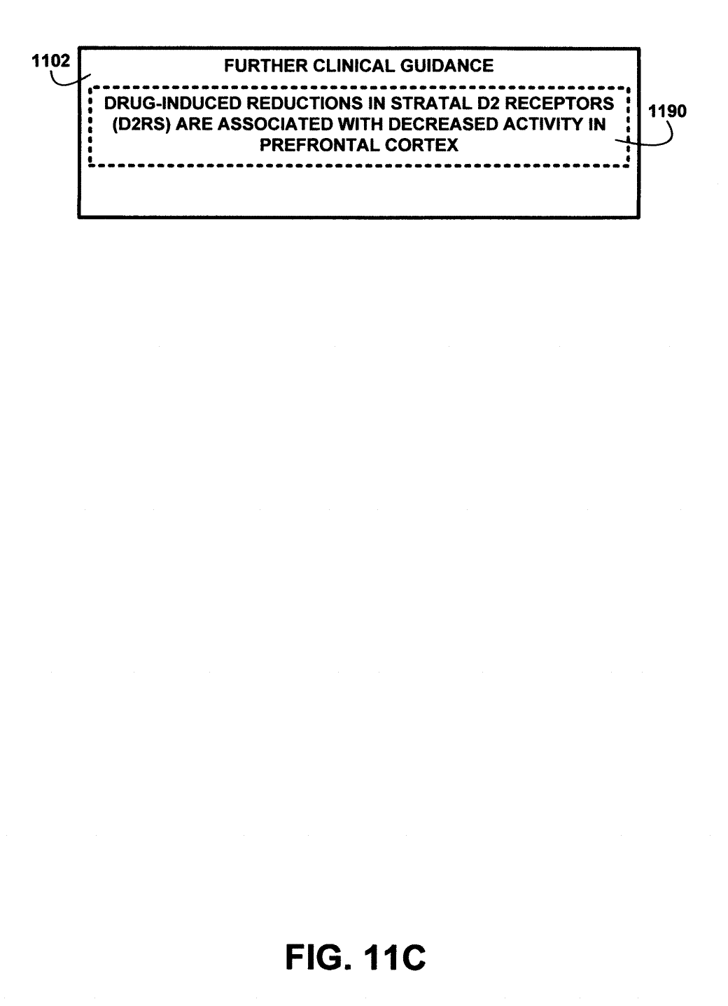

FIG. 11A shows for illustrative purposes only an example of clinical guidance of one embodiment. FIG. 11A shows the PDMPA system 1000 of FIG. 10 using the PDMPA system network 1001 of FIG. 10 to provide clinical guidance 1100 to health care professionals wherein clinical guidance can be viewed in a PDMPA interactive display 1110. The clinical guidance 1100 can include education on the opioid addict susceptible classifications, behaviors and drug use patterns 1120, education on the significance of genetic and toxicology reported conditions that serve as red flags to probable opioid addiction 1130, and patterns of drug use including opioids leading to a heroin addiction 1140. Statistical findings and publications on opioid use and heroin addiction—to alert physicians of additional warning signs 1150. For example people who are addicted to alcohol are 2 times, are addicted to marijuana are 3 times, are addicted to cocaine are 15 times, and are addicted to Rx opioid painkillers are 40 times more likely to be addicted to heroin. This information from the PDMPA system 1000 of FIG. 10 can educate and remind physicians of the critical need to reduce if not eliminate the use of opioid painkillers. Clinical guidance information can be printed and displayed in physician's offices and examination rooms to educate patients on how their current addition could make them more susceptible to a worse addiction to motivate them to seek help and better understand why their doctor is prescribing non-opioid medications to deal with their current or persistent pain issues. Physicians are advised to prescribe the lowest effective dose, only in the quantity required for each patient 1160. Physicians are requested to support the use of FDA-approved MAT options methadone, buprenorphine, and naltrexone in patients with addiction to prescription opioid painkillers or heroin 1170 of one embodiment.

### Additional Clinical Guidance:

FIG. 11B shows for illustrative purposes only an example of additional clinical guidance of one embodiment. FIG. 11B shows additional clinical guidance 1101. Additional clinical guidance 1101 includes requesting physicians to ask patients about past/current drug and alcohol use before considering treatment with an opioid . . . then query the PDMPA system on test results, past medical conditions and treatments and any law enforcement incidents involving drugs to double check the patient past/current drug and alcohol use looking for warning incidents and red and yellow alert status conditions 1180. Physicians are advised to ensure optimal postoperative pain management, guideline recommendations support the use of multimodal analgesic techniques that improve pain control and reduce the need for opioid analgesia, ultimately reducing the risk of opioid-related adverse events 1182. Physicians are made aware of a prospective cohort study of patients undergoing bone or soft tissue procedures wherein virtually all patients received a postoperative analgesic prescription for 30 tablets and on average 19 tablets, left over opioid pills, were left unused per prescription that could potentially become a source for diversion 1184. Physicians are educated on other studies which show the opioid addiction problem to systemic in what are considered to be normal post-surgical opioid prescriptions which can lead to unintended long-term use as shown in a study of patients which showed after one year one-third of all patients were still using opioids, 18% of patients who had not used opioids before surgery were still using opioids and a study of older patients (>65 years) 10.3% were still taking opioids one year later 1186 of one embodiment.

### Further Clinical Guidance:

FIG. 11C shows for illustrative purposes only an example of further clinical guidance of one embodiment. FIG. 11C shows further clinical guidance 1102. The further clinical guidance 1102 provides physicians and patients with an education of the damage associated with drug use for example drug-induced reductions in Stratal D2 Receptors (D2Rs) are associated with decreased activity in prefrontal cortex 1190. The clinical guidance can be printed at a doctor's office and can be posted in a physician's office and examination rooms to educate patients on the risk to their personal health associated with drug abuse.

### Program Administrators PDMPA System Network Interactions:

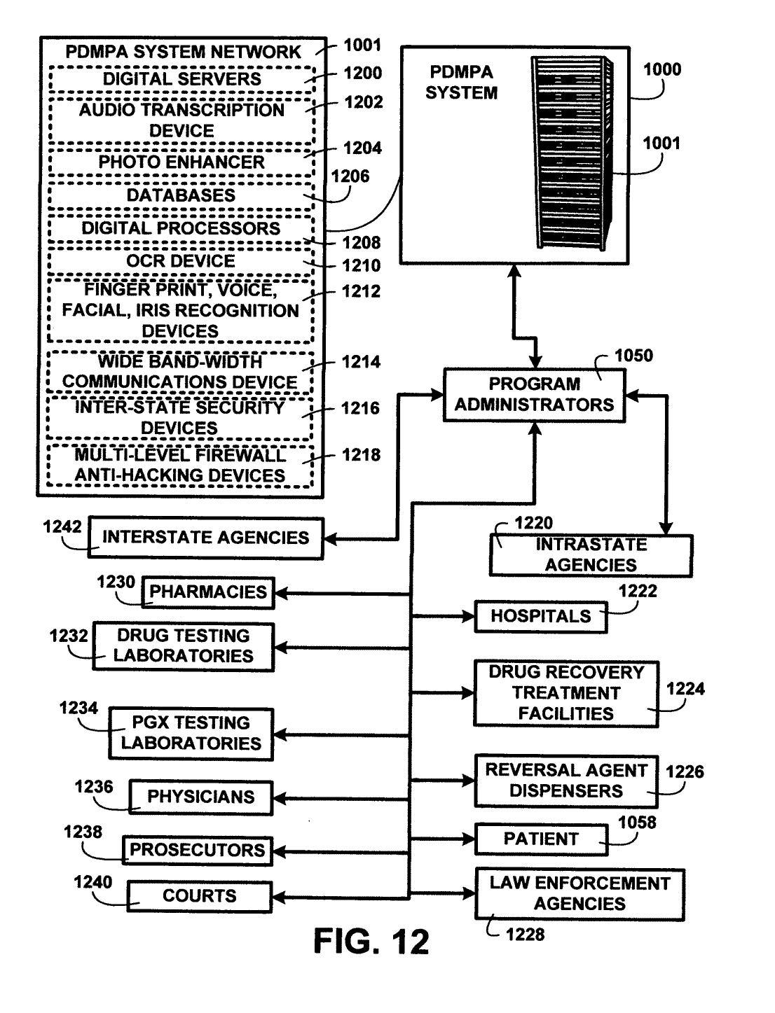

FIG. 12 shows for illustrative purposes only an example of program administrators PDMPA system network interactions of one embodiment. FIG. 12 shows the PDMPA system 1000 using the PDMPA system network 1001 including digital servers 1200, audio transcription device 1202, photo enhancer 1204, databases 1206, digital processors 1208, OCR device 1210, finger print, voice, facial, iris recognition devices 1212, wide band-width communications device 1214, interstate security devices 1216, and multi-level firewall anti-hacking devices 1218. The program administrators 1050 use the PDMPA system 1000 and PDMPA system network 1001 to interact with various entities involved with drug abuse treatment and enforcement against drug related criminal activities.

Entities involved with drug abuse treatment and enforcement against drug related criminal activities include intrastate Agencies 1220, hospitals 1222, drug recovery treatment facilities 1224, reversal agent dispensers 1226, patient 1058, law enforcement agencies 1228, pharmacies 1230, and drug testing laboratories 1232, PGX testing laboratories 1234, physicians 1236, prosecutors 1238, courts 1240, and interstate Agencies 1242. The PDMPA system 1000 provides the vehicle and means for cooperative activities of the entities described above to reduce and possibly end the opioid epidemic currently building in this and other countries of one embodiment.

### Law Enforcement Agency PDMPA System Network Interactions:

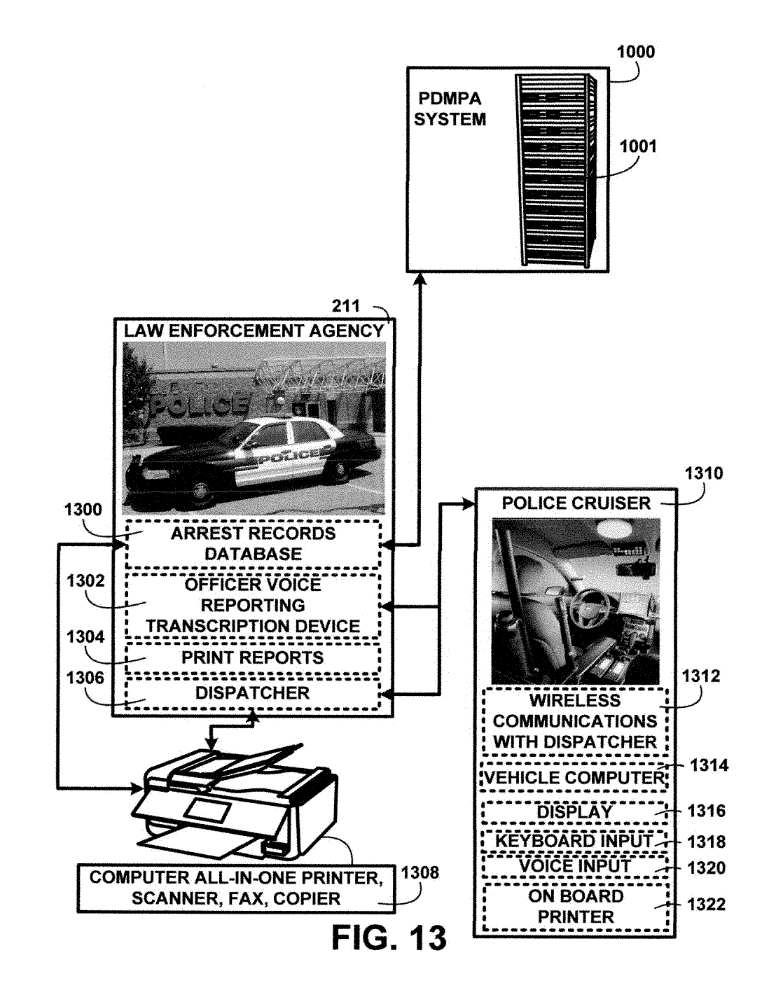

FIG. 13 shows for illustrative purposes only an example of law enforcement agency PDMPA system network interactions of one embodiment. FIG. 13 shows the PDMPA system 1000 using the PDMPA system network 1001 for interactions with a law enforcement agency 211. The law enforcement agency 211 submits data from an arrest records database 1300 to the PDMPA system network 1001 where the data is recorded in databases 1206 of FIG. 12 and analyzed using digital processors 1208 of FIG. 12. The recorded and analyzed data is then available to a law enforcement agency 211 along with data from other entities and agency to provide all available information to identify and arrest suspect using the evidence processed through the PDMPA system 1000 with their current investigative efforts.

The law enforcement agency 211 station can be equipped with an officer voice reporting transcription device 1302 to add to a new or existing file on a suspect or observed activity and then recorded in the law enforcement agency 211 arrest records database 1300 and automatically submitted to the PDMPA system network 1001. The law enforcement agency 211 can print reports 1304 using data requested from the PDMPA system 1000 and transmit that data through a dispatcher 1306 to an officer. The transmission of the data can be to a printer, fax or scanned and emailed to the officer using for example a computer all-in-one printer, scanner, fax, copier 1308.

The dispatcher 1306 can transmit data to and receive data from an officer in a police cruiser 1310 using wireless communications with dispatcher 1312, a vehicle computer 1314 with a display 1316 and an on board printer 1322. The officer can transmit data to the dispatcher 1306 using keyboard input 1318 and a voice input 1320 device which will be transcribed by the officer voice reporting transcription device 1302 to create a written report and possibly be used by the officer for his reports of one embodiment.

### Health Care Industry PDMPA System Network Interactions:

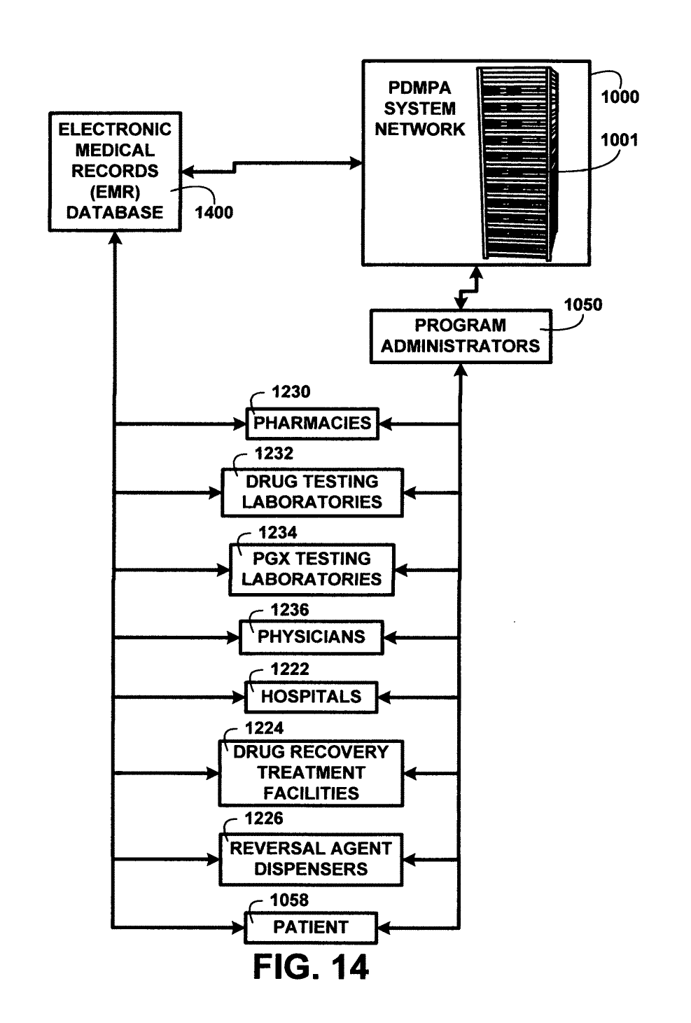

FIG. 14 shows for illustrative purposes only an example of health care industry PDMPA system network interactions of one embodiment. FIG. 14 shows the PDMPA system 1000 and PDMPA system network 1001 being used by program administrators 1050 in performing interactions with the health care industry related to drug abuse and criminal activities. The PDMPA system 1000 is used to process submitted data from pharmacies 1230, drug testing laboratories 1232, PGX testing laboratories 1234, physicians 1236, hospitals 1222, drug recovery treatment facilities 1224, reversal agent dispensers 1226, and patient 1058. The PDMPA system 1000 can also gather data using a connection from the PDMPA system network 1001 to an electronic medical records (EMR) database 1400 of one embodiment.

### Judicial System PDMPA System Network Interactions:

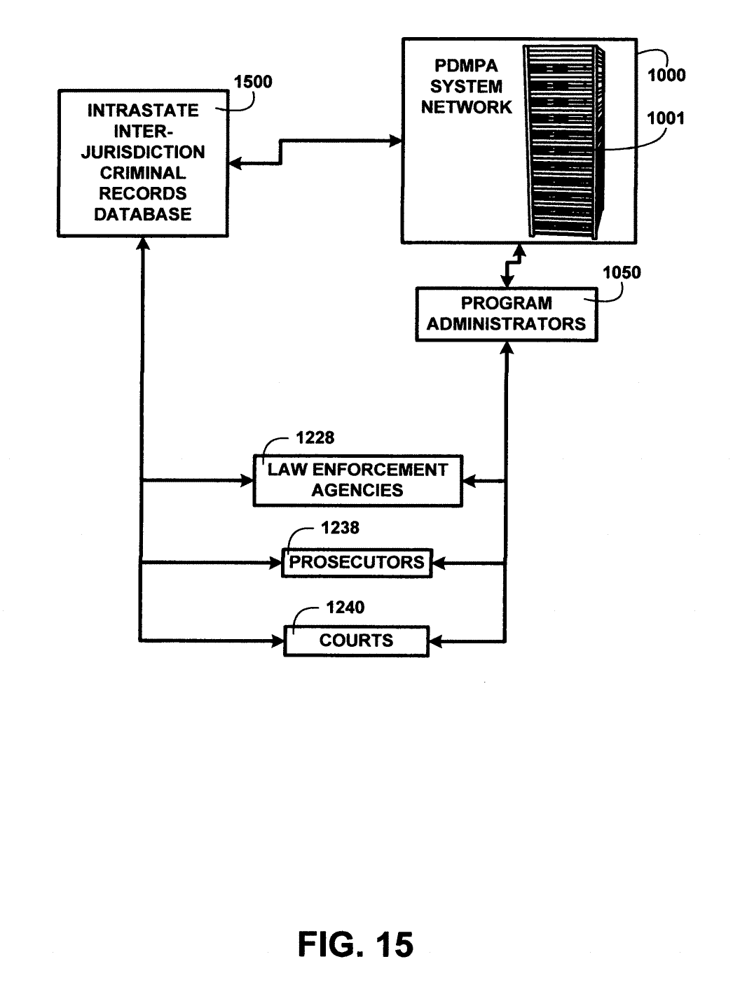

FIG. 15 shows for illustrative purposes only an example of judicial system PDMPA system network interactions of one embodiment. FIG. 15 shows the PDMPA system 1000 and PDMPA system network 1001 being used by program administrators 1050 in performing interactions with the judicial system. The judicial system consists of law enforcement agencies 1228, prosecutors 1238, and courts 1240. Data is submitted to the PDMPA system 1000 through a connection from the PDMPA system network 1001 to an intrastate inter-jurisdiction criminal records database 1500 of one embodiment.

### Interstate Cooperative Data Analysis Sharing:

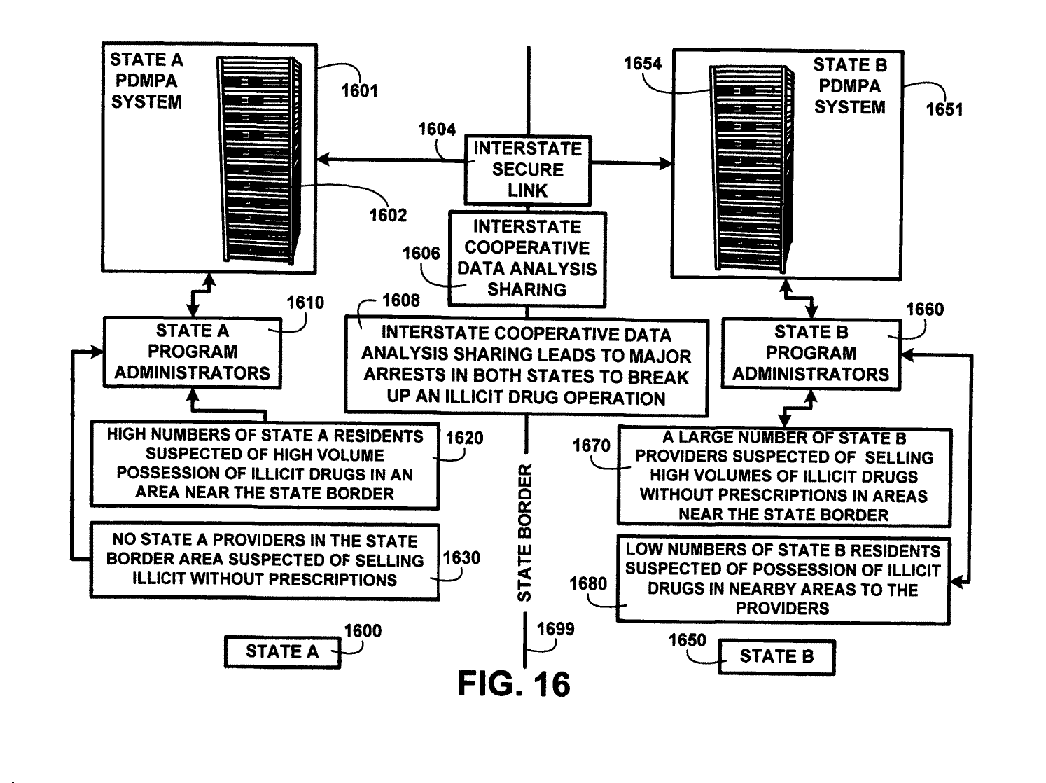

FIG. 16 shows for illustrative purposes only an example of interstate cooperative data analysis sharing of one embodiment. FIG. 16 shows state A 1600 using a state A PDMPA system 1601 and state A PDMPA system network 1602. State A program administrators 1610 operate the state A PDMPA system 1601 including the analysis of the data submitted and process through the state A PDMPA system network 1602.

In this example the State A program administrators 1610 find the analyzed data indicates an anomaly where high numbers of state A residents suspected of high volume possession of illicit drugs in an area near the state Border 1620, yet no state A providers in the state Border area suspected of selling illicit without prescriptions 1630.

State B 1650 also employs a state B PDMPA system 1651 which is operated by state B program administrators 1660. The state B program administrators 1660 see a state B PDMPA system network 1654 produced analysis where a large number of state B providers suspected of selling high volumes of illicit drugs without prescriptions in areas near the state Border 1670, but low numbers of state B residents suspected of possession of illicit drugs in nearby areas to the providers 1680.

State A 1600 and State B 1650 agree to form interstate cooperative data analysis sharing 1606 using an interstate secure link 1604 that crosses their adjacent state Border 1699. Interstate cooperative data analysis sharing 1606 allows both states to more accurately assess where in each state possible illegal activities are taking place that lead to drug abuse. The data anomalies viewed by each state's program administrators show when the shared data analyses are viewed in number tables and graphically as produced by the PDMPA systems it becomes clear cross border sells and purchases are taking place. Without the interstate cooperative data analysis sharing 1606 of the PDMPA systems data analysis 160 of FIG. 1 it would have been difficult to gain arrest evidence and arrest both states offenders.

The cooperative planning on coordinated surveillance and timing of arrests could be more easily planned and all suspects involved better identified by both team of law enforcement agencies using the PDMPA systems complete data information, analysis and correlations. In this example interstate cooperative data analysis sharing leads to major arrests in both states to break up an illicit drug operation 1608 of one embodiment.

In one embodiment a method includes operating at least one prescription drug management program accountability method and system including at least one prescription drug management program accountability method and system network for creating a centralized external prescription documenting system; inputting, analyzing and monitoring testing and prescription activity and lab testing results using at least one prescription drug management program accountability method and system network; receiving lab testing data and integrating the lab testing information into the prescription drug management program accountability method and system patient data; integrating and monitoring prescription activity including pharmacy prescriptions for each patient prescribed a schedule ii, iii and iv drug by having electronic integration of pharmacy data from all dispensing pharmacies and dispensing entities; creating automatically a report and providing clinical guidance to a practitioner using the prescription drug management program accountability method and system including warnings and alert status on risk assessments of opioid drug abuse and risk assessment on addiction; using a prescription drug management program accountability method and system summary report and graphical mapping to find hot beds of illicit drug activity; and using the prescription drug management program accountability method and system for program administrator prescription drug management program accountability method and system network interactions with at least one law enforcement agency, at least one health care industry, at least one judicial system, and at least one interstate cooperative data analysis sharing for reducing illicit drug criminal activity and illicit and prescription drug abuse including opioid addiction. Using the prescription drug management program accountability method and system for program administrator prescription drug management program accountability method and system network interactions with at least one law enforcement agency, and at least one judicial system includes law enforcement illicit drug related arrests reporting and judicial prosecution illicit drug related conviction reporting. Inputting, analyzing and monitoring testing and prescription activity and lab testing results using at least one prescription drug management program accountability method and system network includes pharmacy illicit drug prescriptions filled reporting, drug testing laboratory reporting, PGX testing laboratory reporting, urine testing and drug recovery treatment facility reporting. Creating automatically a report and providing clinical guidance to a practitioner using the prescription drug management program accountability method and system includes education for a practitioner and patients. Using the prescription drug management program accountability method and system for program administrator prescription drug management program accountability method and system network interactions with at least one health care industry includes displaying interactive physician patient prescription alert analysis reporting query response with a warning alert status. Using the prescription drug management program accountability method and system for program administrator prescription drug management program accountability method and system network interactions with at least one health care industry includes recording a physician acknowledgement of review of patient prescription alert warning status analysis reporting query response, wherein a prescribing physician can make an informed decision of the appropriateness of issuing a new prescription to this patient in an effort to control the prescribing and dispensing of illicit drugs. Using the prescription drug management program accountability method and system for program administrator prescription drug management program accountability method and system network interactions with at least one interstate cooperative data analysis sharing includes using an interstate secure link for interstate cooperative data analysis sharing between more than one state to facilitate making major arrests for breaking up illicit drug operations. Operating at least one prescription drug management program accountability method and system network including at least one finger print, voice, facial, iris recognition device is configured for identifying a user login. Operating at least one prescription drug management program accountability method and system including at least one prescription drug management program accountability method and system network includes using at least one digital server, at least one audio transcription device, at least one photo enhancer, at least one database, at least one digital processor, at least one OCR device, at least one finger print, voice, facial, iris recognition device, at least one wide band-width communications device, at least one intrastate security device, at least one interstate security device, and at least one multi-level firewall anti-hacking devices. Creating automatically a report including warnings and alert status on risk assessments of opioid drug abuse and risk assessment on addiction includes the physician acknowledgement that the physician has read and noted the warnings and alerts for accountability on an informed decision of the appropriateness of issuing a new opioid medication prescription to a patient in an effort to control prescribing and dispensing of illicit drugs including opioids.

In another embodiment an apparatus includes at least one prescription drug management program accountability system network to create a centralized external prescription documenting system; at least one digital server with at least one digital processor and with at least one application program interface; at least one prescription drug management program accountability system database to store data from the at least one prescription drug management program accountability system network; at least one audio transcription device; at least one photo enhancer; at least one OCR device; at least one each of a finger print recognition device, voice recognition device, facial recognition device, iris recognition device; at least one wide band-width communications device; at least one GPS mapping device; at least one printing device; at least one digital document scanner device; at least one intrastate security device; at least one interstate security device; and at least one multi-level firewall anti-hacking device. Wherein the at least one prescription drug management program accountability database is configures to receive data inputted automatically from lab testing entities databases including from entities that perform blood tests, urine tests, pharmacogenetic PGX tests, drug tests and other testing as physicians deemed appropriate. Wherein the at least one digital server with at least one digital processor and with at least one application program interface is configured to receive, transmit, analyze, perform calculations, process, and retrieve data from multiple digital data communications connections to multiple digital devices with dissimilar operating systems. Where in the at least one each of a finger print recognition device, voice recognition device, facial recognition device, iris recognition device is configured for identifying a user login. Where in the centralized external prescription documenting system is configured to provide a physician with data displayed on a user digital device in a doctor's office relating to a patient that has been analyzed to produce warning incidents and alerts relating to past and recent prescriptions, arrest and convictions, lab testing results, drug and addiction risk assessments, and clinical guidance to make an informed decision of the appropriateness of issuing a new opioid medication prescription to a patient in an effort to control prescribing and dispensing of illicit drugs including opioids.

In yet another embodiment is a process for analyzing and integrating data including receiving, storing and analyzing lab testing data including toxicology drug testing, pharmacogenetic testing (PGX), blood testing, urine testing and other drug use related lab testing; integrating the lab testing data into a prescription drug management program accountability system network patient data database; processing the lab testing data for a patient includes integrating, monitoring and analyzing pharmacy prescriptions data from all dispensing pharmacies and dispensing entities; wherein analyzing the lab testing data and the integrated pharmacy prescriptions data to assess a patient's risk of drug abuse and addiction and create warnings and alerts based on the risk assessments; processing includes creating automatically reports and providing the reports and clinical guidance to a practitioner using the prescription drug management program accountability system network that includes warnings and alerts on opioid drug abuse and addictive risk assessment to make an informed decision of the appropriateness of issuing a new opioid medication prescription to a patient in an effort to control prescribing and dispensing of illicit drugs including opioids. Wherein warnings and alerts on opioid drug abuse and addictive risk assessment can include a number of warning incidents for a behavior and offense and an alert status letter and color indication of a warning level of risk assessment. Wherein analyzing toxicology drug testing, blood testing, urine testing and other drug use related lab testing can produce a comprehensive assessment of a patient's past and current drug use and produce a warning of current opioid addiction risk assessment for current pain medication prescribing and dispensing. Wherein processing produced warnings and alerts on opioid drug abuse and addictive risk assessment includes the physician acknowledgement that the physician has read and noted the warnings and alerts for accountability on an informed decision of the appropriateness of issuing a new opioid medication prescription to a patient in an effort to control prescribing and dispensing of illicit drugs including opioids. Wherein providing the clinical guidance can include education on how to interpret patient data based on the patient past and current behavior including past arrest for an illicit drug, and filling of opioid prescriptions from multiple prescribers, paying cash and other indications of a drug abuse behavior.

The foregoing has described the principles, embodiments and modes of operation of the embodiments. However, the embodiments should not be construed as being limited to the particular embodiments discussed. The above described embodiments should be regarded as illustrative rather than restrictive, and it should be appreciated that variations may be made in those embodiments by workers skilled in the art without departing from the scope of the present invention as defined by the following claims.

What is claimed is:

1. A method, comprising:
providing an opioid drug abuse and risk assessment behavioral mental health screen to a patient needing an opioid prescription within a prescription drug management accountability system;
electronically connecting the prescription drug management accountability system to a centralized prescription drug monitoring system that is electronically connected to licensed pharmacies and licensed dispensing entities; and
collecting pharmacy prescription information for the patient from the centralized prescription drug monitoring system and producing a report with information on opioid prescriptions of the patient.

2. The method of claim 1, wherein using the prescription drug management program accountability method and system for program administrator prescription drug management program accountability method and system network interactions with at least one law enforcement agency, and at least one judicial system includes law enforcement illicit drug related arrests reporting and judicial prosecution illicit drug related conviction reporting.

3. The method of claim 1, wherein inputting, analyzing and monitoring testing and prescription activity and lab testing results using at least one prescription drug management program accountability method and system network includes pharmacy illicit drug prescriptions filled reporting, drug testing laboratory reporting, PGX testing laboratory reporting, urine testing and drug recovery treatment facility reporting.

4. The method of claim 1, wherein creating automatically a report and providing clinical guidance to a practitioner using the prescription drug management program accountability method and system includes education for a practitioner and patients.

5. The method of claim 1, wherein using the prescription drug management program accountability method and system for program administrator prescription drug management program accountability method and system network interactions with at least one health care industry includes displaying interactive physician patient prescription alert analysis reporting query response with a warning alert status.

6. The method of claim 1, wherein using the prescription drug management program accountability method and system for program administrator prescription drug management program accountability method and system network interactions with at least one health care industry includes recording a physician acknowledgement of review of patient prescription alert warning status analysis reporting query response, wherein a prescribing physician can make an informed decision of the appropriateness of issuing a new prescription to this patient in an effort to control the prescribing and dispensing of illicit drugs.

7. The method of claim 1, wherein using the prescription drug management program accountability method and system for program administrator prescription drug management program accountability method and system network interactions with at least one interstate cooperative data analysis sharing includes using an interstate secure link for interstate cooperative data analysis sharing between more than one state to facilitate making major arrests for breaking up illicit drug operations.

8. The method of claim 1, wherein operating at least one prescription drug management program accountability method and system network including at least one finger print, voice, facial, iris recognition device is configured for identifying a user login.

9. The method of claim 1, wherein operating at least one prescription drug management program accountability method and system including at least one prescription drug management program accountability method and system network includes using at least one digital server, at least one audio transcription device, at least one photo enhancer, at least one database, at least one digital processor, at least one OCR device, at least one finger print, voice, facial, iris recognition device, at least one wide band-width communications device, at least one intrastate security device, at least one interstate security device, and at least one multi-level firewall anti-hacking devices.

10. The method of claim 1, wherein creating automatically a report including warnings and alert status on risk assessments of opioid drug abuse and risk assessment on addiction includes the physician acknowledgement that the physician has read and noted the warnings and alerts for accountability on an informed decision of the appropriateness of issuing a new opioid medication prescription to a patient in an effort to control prescribing and dispensing of illicit drugs including opioids.

11. An apparatus, comprising:
a prescription drug management accountability system;
an opioid drug abuse and risk assessment behavioral mental health test integrated within the prescription drug management accountability system for screening a patient that needs an opioid prescription;
a centralized prescription drug monitoring system that is electronically connected to licensed pharmacies and licensed dispensing entities, wherein the centralized prescription drug monitoring system is electronically connected to the prescription drug management accountability system; and
wherein the centralized prescription drug monitoring system collects pharmacy prescription information for the patient and produces a report with information on opioid prescriptions of the patient.

12. The apparatus of claim 11, wherein the at least one prescription drug management program accountability database is configures to receive data inputted automatically from lab testing entities databases including from entities that perform blood tests, urine tests, pharmacogenetic PGX tests, drug tests and other testing as physicians deemed appropriate.

13. The apparatus of claim 11, wherein the at least one digital server with at least one digital processor and with at least one application program interface is configured to receive, transmit, analyze, perform calculations, process, and retrieve data from multiple digital data communications connections to multiple digital devices with dissimilar operating systems.

14. The apparatus of claim 11, where in the at least one each of a finger print recognition device, voice recognition device, facial recognition device, iris recognition device is configured for identifying a user login.

15. The apparatus of claim 11, where in the centralized external prescription documenting system is configured to provide a physician with data displayed on a user digital device in a doctor's office relating to a patient that has been analyzed to produce warning incidents and alerts relating to past and recent prescriptions, arrest and convictions, lab testing results, drug and addiction risk assessments, and clinical guidance to make an informed decision of the appropriateness of issuing a new opioid medication prescription to a patient in an effort to control prescribing and dispensing of illicit drugs including opioids.

16. A process for analyzing and integrating data, comprising:
receiving, storing and analyzing lab testing data including toxicology drug testing, pharmacogenetic testing (PGX), blood testing, urine testing and other drug use related lab testing;
integrating the lab testing data into a prescription drug management program accountability system network patient data database;
processing the lab testing data for a patient includes integrating, monitoring and analyzing pharmacy prescriptions data from all dispensing pharmacies and dispensing entities;
wherein analyzing the lab testing data and the integrated pharmacy prescriptions data to assess a patient's risk of drug abuse and addiction and create warnings and alerts based on the risk assessments;
processing includes creating automatically reports and providing the reports and clinical guidance to a practitioner using the prescription drug management program accountability system network that includes warnings and alerts on opioid drug abuse and addictive risk assessment to make an informed decision of the appropriateness of issuing a new opioid medication prescription to a patient in an effort to control prescribing and dispensing of illicit drugs including opioids.

17. The process for analyzing and integrating data of claim 16, wherein warnings and alerts on opioid drug abuse and addictive risk assessment can include a number of warning incidents for a behavior and offense and an alert status letter and color indication of a warning level of risk assessment.

18. The process for analyzing and integrating data of claim 16, wherein analyzing toxicology drug testing, blood testing, urine testing and other drug use related lab testing can produce a comprehensive assessment of a patient's past and current drug use and produce a warning of current opioid addiction risk assessment for current pain medication prescribing and dispensing.

19. The process for analyzing and integrating data of claim 16, wherein processing produced warnings and alerts on opioid drug abuse and addictive risk assessment includes the physician acknowledgement that the physician has read and noted the warnings and alerts for accountability on an informed decision of the appropriateness of issuing a new opioid medication prescription to a patient in an effort to control prescribing and dispensing of illicit drugs including opioids.

20. The process for analyzing and integrating data of claim 16, wherein providing the clinical guidance can include education on how to interpret patient data based on the patient past and current behavior including past arrest for an illicit drug, and filling of opioid prescriptions from multiple prescribers, paying cash and other indications of a drug abuse behavior.
